(19) United States
(12) Patent Application Publication
(10) Pub. No.: US 2007/0161998 A1
(43) Pub. Date: Jul. 12, 2007

(54) Instruments and Methods For Manipulating A Spinal Rod

(76) Inventor: Dale Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/550,852

(22) Filed: Oct. 19, 2006

Related U.S. Application Data
(60) Provisional application No. 60/731,072, filed on Oct. 28, 2005.

(51) Int. Cl. A61F 2/30

(52) U.S. Cl. 606/61

(57) ABSTRACT

An instrument for manipulating a spinal rod relative to a bone anchor may include a first shaft and a first finger and a second finger provided at a distal end of the first shaft. The first finger may be spaced apart a distance from the second finger approximate to the outer diameter of the receiver member of the bone anchor. Each of the first finger and the second finger may include a projection extending along the length of the finger for engaging a groove provided on the receiver member of the bone anchor. The instrument may also include a second shaft that is movable relative to the first shaft in a direction parallel to the longitudinal axis of the first shaft to manipulate the spinal rod relative to the receiver member of the bone anchor.

### CROSS-REFERENCE TO RELATED APPLICATIONS

This application claims priority to U.S. Provisional Patent Application No. 60/731,072 filed Oct. 28, 2005, which is incorporated herein by reference.

### Background

Spinal systems may be used in orthopedic surgery to align and/or fix a desired relationship between adjacent vertebrae. Such systems typically include a spinal connection element, such as a relatively rigid fixation rod or plate, a relatively flexible tether or cable, or a dynamic connection element that is coupled to adjacent vertebrae by attaching the element to various anchoring devices, such as hooks, bolts, wires, or screws. The spinal connection element can have a predetermined contour that has been designed according to the properties of the target implantation site, and once installed, the spinal connection element holds the vertebrae in a desired spatial relationship, either until desired healing or spinal fusion has taken place, or for some longer period of time.

Spinal connection elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal connection element receiving element, which, in spinal rod applications, is usually in the form of a U-shaped slot formed in the head for receiving the rod. A set-screw, plug, cap or similar type of closure mechanism, is used to lock the rod into the rod-receiving portion of the pedicle screw. In use, the shank portion of each screw is then threaded into a vertebra, and once properly positioned, a spinal rod or other connection element is seated through the rod-receiving portion of each screw and the rod is locked in place by tightening a cap or similar type of closure mechanism to securely interconnect each screw and the spinal rod. Other anchoring devices also include hooks and other types of bone screws.

While current spinal systems have proven effective, difficulties have been encountered in mounting rods, or other spinal connection elements, into the rod-receiving portion of various fixation devices. In particular, it can be difficult to align and seat the rod into the rod receiving portion of adjacent fixation devices due to the positioning and rigidity of the vertebra into which the fixation device is mounted. Thus, the use of a spinal rod approximator, also referred to as a spinal rod reducer, is often required in order to grasp the head of the fixation device, and reduce the rod into the rod-receiving portion of the fixation device.

While several rod approximators are known in the art, some tend to be difficult and very time-consuming to use. Accordingly, there is a need for an improved rod approximator and methods for seating a spinal rod in a rod-receiving portion of one or more spinal implants.

### SUMMARY

Disclosed herein are instruments and methods for engaging a bone anchor, such as a polyaxial or monoaxial bone screw, and/or manipulating a spinal connection element, such as a spinal rod, relative to the bone anchor. The instruments and methods disclosed herein are particularly suited for vertical alignment of a spinal connection element relative to a bone anchor.

In accordance with one exemplary embodiment, an instrument for manipulating a spinal rod relative to a bone anchor may comprise a first shaft having a longitudinal axis and a first finger and a second finger provided at a distal end of the first shaft. The first finger may be spaced apart a distance from the second finger and the distance between the first finger and the second finger may be approximate to the outer diameter of the receiver member of the bone anchor. Each of the first finger and the second finger may include a projection extending along the length of the finger for engaging a groove provided on the receiver member of the bone anchor. The instrument may also include a second shaft that is movable relative to the first shaft in a direction parallel to the longitudinal axis of the first shaft to manipulate the spinal rod relative to the receiver member of the bone anchor.

In accordance with another exemplary embodiment, a spinal implant and instrument kit may comprise a spinal rod, a bone anchor, and an instrument for manipulating the spinal rod relative to the bone anchor. The bone anchor may have a distal bone engaging portion and a receiving member having a proximal end having a recess for receiving the spinal rod. The receiving member may have a substantially U-shaped cross-section defined by two legs separated by the recess. The receiving member may have a first groove formed on an exterior surface of the proximal end of a first one of the legs and a second groove formed on an exterior surface of the proximal end of a second one of the legs. The instrument may include a first shaft having a longitudinal axis and a first finger and a second finger provided at a distal end of the first shaft. The first finger may be spaced apart a distance from the second finger. The first finger may include a first projection extending along at least a portion of the length of the first finger for engaging the first groove provided on the receiver member of the bone anchor. The second finger may include a second projection extending along at least a portion of the length of the second finger for engaging the second groove provided on the receiver member of the bone anchor. The instrument may also include a second shaft movable relative to the first shaft in a direction parallel the longitudinal axis of the first shaft to manipulate the spinal rod relative to the receiver member of the bone anchor.

In accordance with another exemplary embodiment, a spinal implant and instrument kit may comprise a bone anchor and an instrument for engaging the bone anchor. The bone anchor may have a distal bone engaging portion and a receiving member having a proximal end having a recess for receiving the spinal rod. The receiving member may have a substantially U-shaped cross-section defined by two legs separated by the recess. The receiving member may have a first groove formed on an exterior surface of the proximal end of a first one of the legs and a second groove formed on an exterior surface of the proximal end of a second one of the legs. The instrument may include a shaft having a longitudinal axis and a generally U-shaped distal end defined by a first member spaced apart from a second member. The first member and the second member may be oriented at an angle to the shaft. The first member may include a first projection extending along at least a portion of the length of the first member for engaging the first groove provided on the receiver member of the bone anchor. The second member may include a second projection extending along at least a portion of the length of the second member for engaging the second groove provided on the receiver member of the bone anchor.

### BRIEF DESCRIPTION OF THE FIGURES

These and other features and advantages of the instruments and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1 is perspective view of an exemplary embodiment of the distal end of an instrument engaging a spinal anchor and for manipulating a spinal connection element relative to the bone anchor;

FIG. 2 is a front view of the instrument of FIG. 1, illustrating the instrument engaged to the bone anchor;

FIG. 3 is a front view of another exemplary embodiment of the distal end of an instrument engaging a spinal anchor and for manipulating a spinal connection element relative to the bone anchor;

FIG. 4 is a side view of the distal end of the instrument of FIG. 3, illustrating the instrument engaged to the bone anchor;

FIG. 5 is a perspective view of another exemplary embodiment of the distal end of an instrument engaging a spinal anchor and for manipulating a spinal connection element relative to the bone anchor;

FIG. 6 is a side view of the instrument of FIG. 3, illustrating an exemplary embodiment of an activation mechanism of the instrument;

FIG. 7 is a side view of the instrument of FIG. 3, illustrating another exemplary embodiment of an activation mechanism of the instrument;

FIG. 8 is a top view of the distal end of the instrument of FIG. 1; and

FIG. 9 is a front view of the distal end of the instrument of FIG. 1.

### DETAILED DESCRIPTION OF EXEMPLARY EMBODIMENTS

Certain exemplary embodiments will now be described to provide an overall understanding of the principles of the structure, function, manufacture, and use of the instruments and methods disclosed herein. One or more examples of these embodiments are illustrated in the accompanying drawings. Those of ordinary skill in the art will understand that the instruments and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

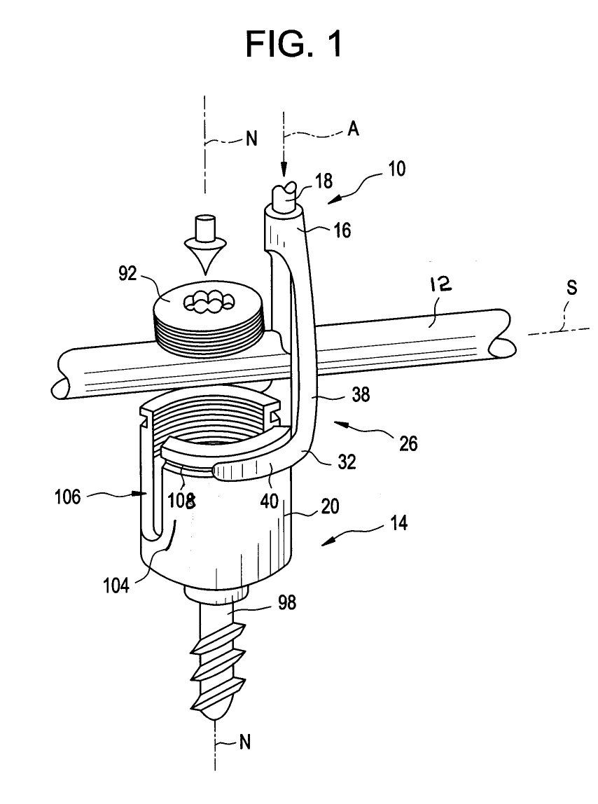

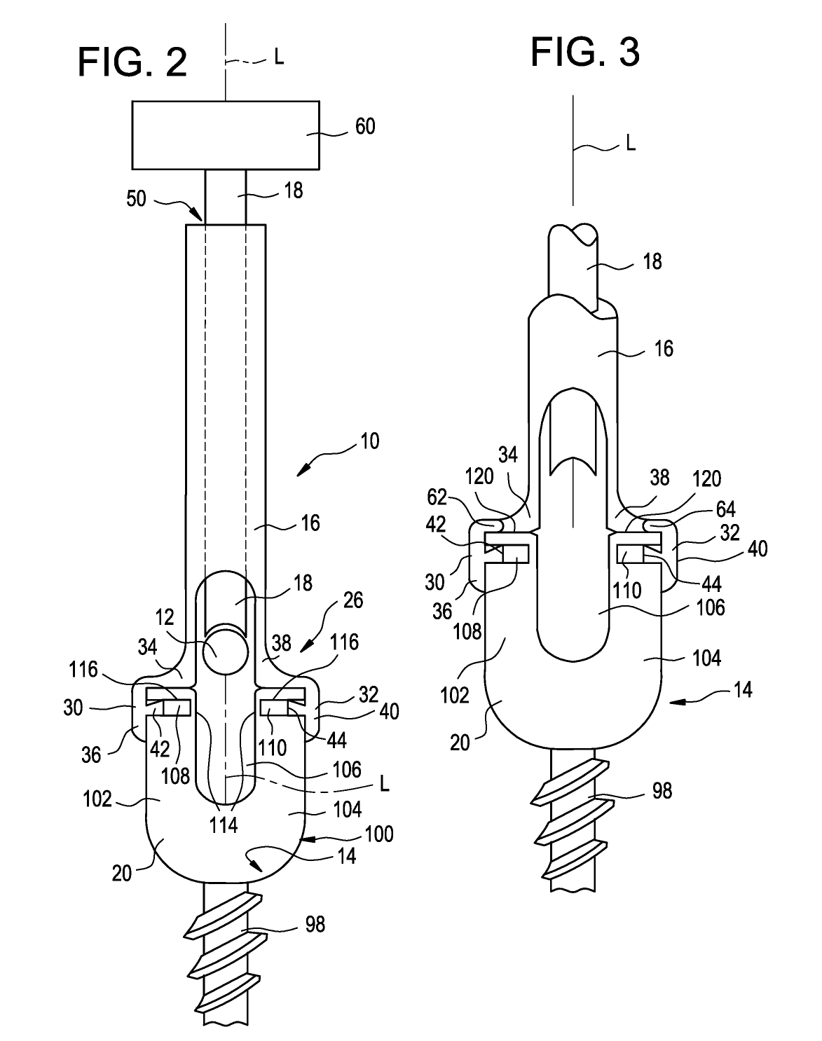

FIGS. 1-2 and 8 illustrate an exemplary embodiment of an instrument 10 for manipulating a spinal connection element 12, such as, for example, a rigid or dynamic spinal rod, a plate, a tether or cable or combinations thereof, relative to a bone anchor 14, such as, for example, a bone screw or hook. The exemplary instrument 10 is particularly suited for vertical adjustment of the spinal connection element 12 relative to the bone anchor 14. The exemplary instrument 10 includes a first shaft 16 that is configured to engage the bone anchor 14 and a second shaft 18 movable relative to the first shaft 16 in a direction parallel to the longitudinal axis L of the first shaft 16, as indicated by arrow A, to manipulate the spinal connection element 12 relative to the receiver member 20 of the bone anchor 14.

As illustrated in the description of the exemplary instrument 10 that follows the spinal connection element is a spinal rod 12 and the bone anchor is a polyaxial bone screw 14, such as a polyaxial pedicle screw. The exemplary bone anchor 14 includes distal bone engaging portion 98 and a receiver member 20 having a proximal end 100 for receiving the spinal connection element 12. The receiver member 20 has a substantially U-shaped cross-section defined by two legs 102, 104 separated by a recess 106 for receiving the spinal connection element 12. The receiver member 20 includes a first groove 108 formed on an exterior surface of the proximal end of the first leg 102 and a second groove 110 formed on the exterior surface of the second leg 104. In the exemplary embodiment, the first groove 108 and the second groove 110 may be generally arcuate in shape and may have a cross section to facilitate insertion of a projection into the groove and/or to inhibit undesirable separation of the projection from the groove. For example, the distal surface 114 and/or the proximal surface 116 of each groove 108, 110 may be oriented at an angle to an orthogonal line which is oriented at approximately perpendicular to the longitudinal axis of the receiver member 20 to facilitate insertion of a projection into the groove and/or retention of the projection in the groove. Exemplary bone anchors having such features are described in U.S. Patent Application Publication No. US 2005/0131408 A1, incorporated herein by reference. One skilled in the art will appreciate that the spinal connection element and the bone anchor are not limited to the illustrated exemplary embodiments. The instrument may be used with any type of spinal connection element and any type of bone anchor.

Continuing to refer to FIGS. 1, 2, 8 and 9, the distal end 26 of the first shaft 16 of the exemplary instrument 10 may be configured to engage the receiver member 20 of a bone anchor 14. The distal end 26 of the first shaft 16 may be generally U-shaped, when viewed from above, defined by first member or first finger 30 spaced apart from a second member or second finger 32. The first finger 30 may include a proximal segment 34 extending generally parallel to the longitudinal axis L of the first shaft 16 and a distal segment 36 extending generally at angle to the longitudinal axis L of the first shaft 18. For example, in the illustrated embodiment, the distal segment 36 is oriented generally perpendicular to the longitudinal axis L of the first shaft 16, although, one skilled in the art will appreciate that other angles also may be suitable. The second finger 32 may be constructed in a manner analogous to the first finger 30. For example, the second finger 32 may include a proximal segment 38 extending generally parallel to the longitudinal axis L of the first shaft 16 and a distal segment 40 extending generally at angle to the longitudinal axis L of the first shaft 18. The distal segments 36, 40 may be oriented at an angle greater than approximately 45° relative to the longitudinal axis L of the first shaft 18. In the illustrated exemplary embodiment, the distal segments 36, 40 are oriented at an angle equal to approximately 90° relative to the longitudinal axis L of the first shaft 18.

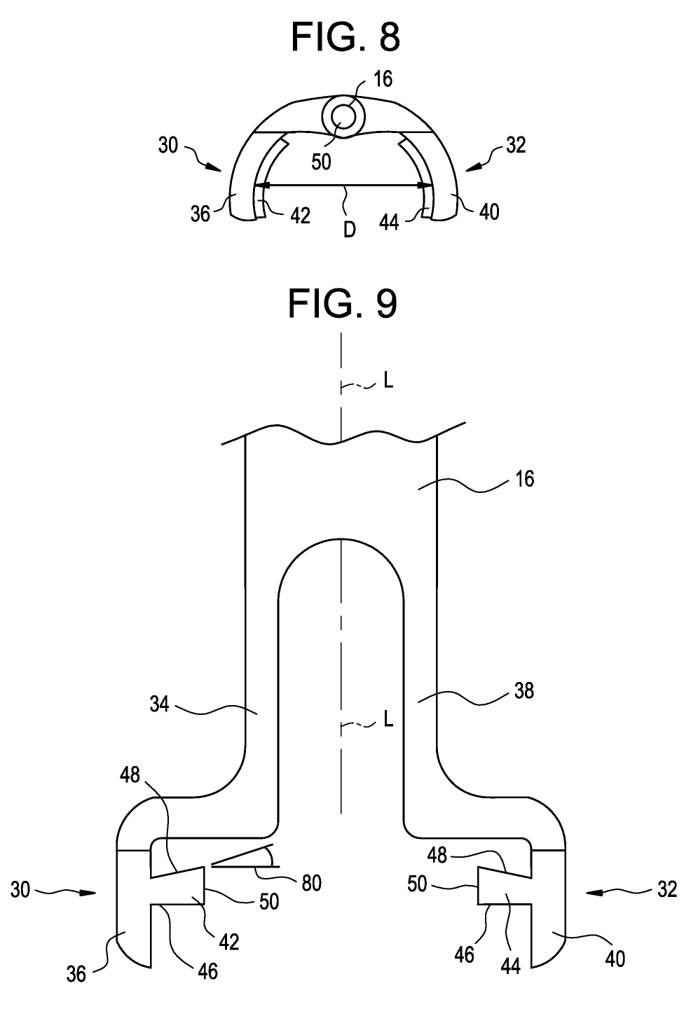

The distal segment 36 of the first finger 30 may be spaced apart from the distal segment 40 of the second finger 32 a distance sufficient to receive the receiver member 20 of the bone anchor 14 between the distal segments. Referring to FIG. 8, for example, the distance D between the distal segment 36 of the first finger 30 and the distal segment 40 of the second finger 32 may be approximate the outer diameter of the receiver member 20 of the bone anchor 14. In certain exemplary embodiments, the distal segments 36, 40 may be flexible and resilient to permit the distal segments 36, 40 to initially flex apart to receive the receiver member 20 and return to an initial position to receive the receiver member between the distal segments 36,40. The distal segment 36 of the first finger 30 and the distal segment 40 of the second finger 32 may be generally arcuate in shape and may have a curvature approximate the curvature of the outer surface of the receiver member 20.

Referring to FIGS. 1, 2, 8 and 9, the distal segments 36, 40 of the first finger 30 and/or the second finger 32 may be configured to engage an opening provided in the bone anchor 14. For example, the distal segment 36 of the first finger 30 may include one or more radially inward facing projections 42 that is sized and shaped to seat within an opening provided in a portion of the receiver member 20 of the bone anchor 14. The size, shape and number of projections can be varied depending on, for example, the opening(s) provided on the bone anchor and type of connection desired. In the illustrated exemplary embodiment, for example, the projection 42 is generally arcuate in shape and has a cross section and a curvature that is complementary to an arcuate groove 108 provided in the receiving member 20 of the exemplary bone anchor 14. In particular, the projection 42 may have a distal surface 46, a proximal surface 48, and a generally radially facing connecting surface 50 that spans between the distal surface 46 and the proximal surface 48, as shown in FIG. 9. In the illustrated embodiment, the distal surface 46 is generally oriented perpendicular to the longitudinal axis L of the instrument 10 and the connecting surface 50 is generally oriented parallel to the longitudinal axis L of the instrument 10 and perpendicular to the distal surface 46. One or both of the proximal surface 48 and the distal surface 46 may be oriented at an angle other than perpendicular to the longitudinal axis L of the instrument 10. For example, the proximal surface 48 may be oriented at an angle B to an orthogonal line 80, which is oriented perpendicular to the longitudinal axis L of the instrument 10. In the exemplary embodiment, the angle B may be approximately 5° to approximately 30° and is preferably approximately 20°. The distal surface 46 and the proximal surface 48 may be oriented at the same angle or, as in the exemplary embodiment, may be oriented at different angles. The distal segment 40 of the second finger 40 may include a projection 44 that is constructed analogously to the projection 42 of the distal segment 36 of the first finger 30.

The projection 42 of the distal segment 36 of the first finger 30 and the projection 44 of the distal segment 40 of the second finger 32 may extend along at least a portion of the length of the respective distal segment 36, 40. In the illustrated embodiment, for example, the projection 42 and the projection 44 have length approximate to the length of the distal segment 36 and distal segment 40, respectively. In alternative exemplary embodiments, one or both of the projections may have a length less than the length of the respective segments. In illustrated exemplary embodiment, the projections are continuous along the length of the respective distal segment. In alternative exemplary embodiments, one or more of the projections may comprise one or more spaced apart projections positioned along a respective distal segment.

Continuing to refer to FIGS. 1, 2, 8, and 9, the second shaft 18 of the exemplary instrument 10 may be movable relative to the first shaft 16 to manipulate the spinal connection element 12 relative to the bone anchor 14. In the illustrated exemplary embodiment, the first shaft 16 is generally tubular in shape have a centrally located lumen 50. At least a portion of the second shaft 18 may be positioned in the lumen 50 of the first shaft 16 and the second shaft 18 may be movable within the lumen 50 of the first shaft to manipulate the spinal connection element 12 relative to the bone anchor 14 when the first shaft 16 is engaged to the bone anchor 14.

The exemplary instrument 10 may include an activation mechanism coupled to the first shaft 16 and the second shaft 18 to effect motion of the second shaft 18 relative to the first shaft 16. For example, the second shaft 18 may include a knob 60 that facilitates motion of the second shaft 18 relative to the first shaft 16. In certain exemplary embodiments, the first shaft 16 and the second shaft 18 may be threadingly engaged to each other. For example, the second shaft 18 may include an external thread that engages and an internal thread provided within the lumen 50 of the first shaft 16 upon rotation of the second shaft 18 relative to the first shaft 16. The knob 60 may facilitate rotation of the second shaft 18 relative to the first shaft 16. The knob 60 and a portion of the exterior surface of the first shaft 16 may include surface features 70 that facilitate gripping of the knob 60 and the first shaft 16. Such surface features may include knurling, sand blasting, or surface coatings.

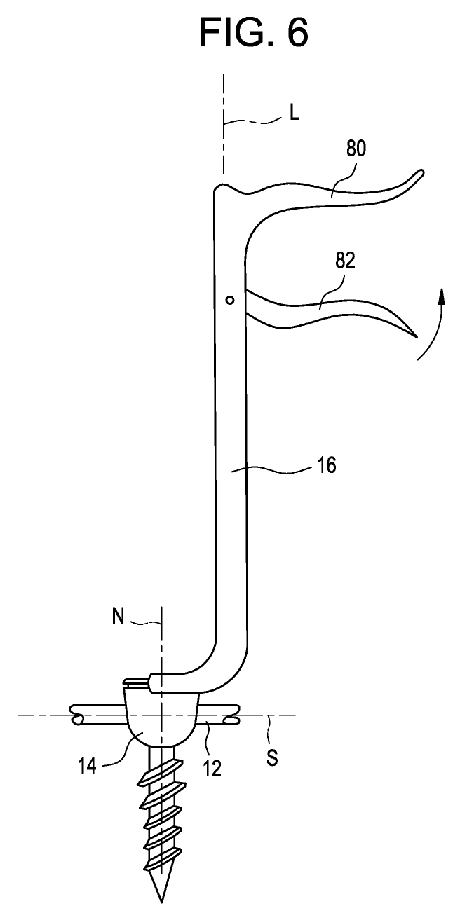

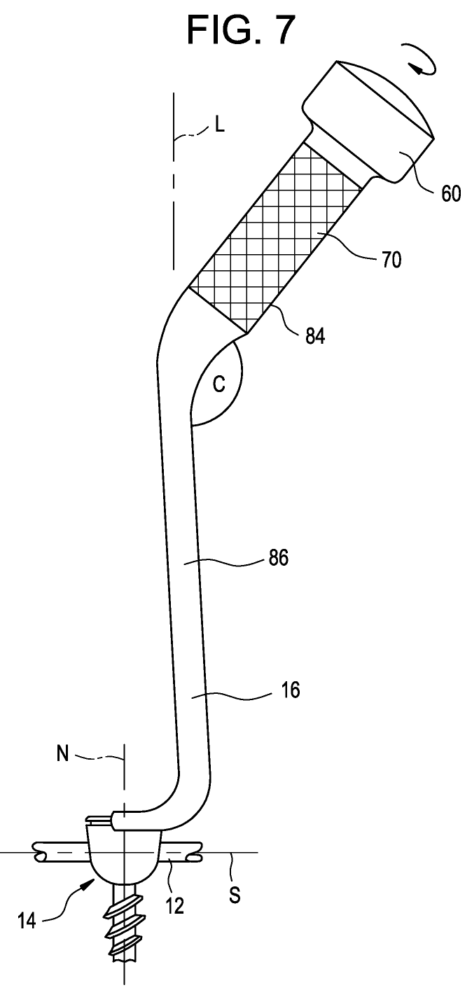

In alternative exemplary embodiments, the activation mechanism of the instrument 10 may include a first handle connected to the first shaft 16 and a second handle connected to the second handle 18. Referring to FIG. 6, for example, a first handle 80 may be connected to the first shaft 16 and a second handle 82 connected to the second shaft 18 may be oriented in a direction perpendicular to the longitudinal axis L of the first shaft 16 and the instrument 10. In the illustrated embodiment, the second handle 18 may be pivotally connected to the second shaft 18 and the first handle 80 may be pivotally connected to the first shaft 16. In alternative embodiments, the first handle 80 also may be pivotally connected to the first shaft 16. A spring may be provided between the first handle 80 and the second handle 82. A locking mechanism, such as a ratchet mechanism, may be provided to fix the first handle 80 relative to the second handle 82. In an alternative exemplary embodiment illustrated in FIG. 7, a proximal segment 84 of the first shaft 16 may be oriented at angle C to the distal segment 86 of the first shaft 16. The angle C may be any angle equal to or greater than approximately 180° and in certain exemplary embodiments is between approximately 180° and approximately 45°. In the exemplary embodiment illustrated in FIG. 7, the second shaft 18 may be jointed to facilitate rotation of the shaft 18 through the angle in the first shaft 16. For example, the second shaft 18 may include two shafts connected by gears, such as a worm, or other mechanisms that permits rotation and transmission of the reduction force through the angle.

Referring to FIGS. 1, 6, and 7, the longitudinal axis L of the first shaft 16 of the exemplary instruments is offset from the neutral axis N of the bone anchor 14 in a direction parallel to the axis of the spinal connection element 12 when the distal end 26 of the first shaft 16 is a engaged to the bone anchor 14. A closure mechanism delivery instrument may be provided for the delivery of a closure mechanism 92, for example, a set screw or the like, to the bone anchor 14 to secure the spinal connection element 12 relative to the bone anchor 12 after alignment of the spinal connection element 12. The closure mechanism delivery instrument may be a screwdriver having a distal end with external lobes for engaging the closure mechanism.

The components of the exemplary instrument 10 may be made from any material suitable for use in vivo, including, for example, metals such as stainless steel and titanium, polymers, or composites thereof. The components of the exemplary instrument 10 may be constructed of the same or different materials.

In use, the exemplary instrument 10 may be employed to engage a bone anchor and to adjust the position of a spinal connection element 12 relative to the bone anchor. The distal end 26 of the first shaft 16 may be engaged with the bone anchor 14 by moving the distal end 26 in a direction parallel to the longitudinal axis S of the spinal connection element 12. The receiver member 20 may be received between the first finger 30 and the second finger 32 and the projections 42, 44 may be positioned within the grooves 108, 110, respectively, to couple the first shaft 16 to the receiver member 20 of the bone anchor 14. Once the first shaft 16 is coupled to the receiver member 20 of the bone anchor 14, the second shaft 18 may be advanced distally in the direction indicated by arrow A in FIG. 1 into contact with spinal connection element 12. Further advancement of the second shaft 18 toward the bone anchor 14 advances the spinal connection element 12 toward the bone anchor 14 until the spinal connection element 12 is seated in slot 106 of the receiver member 20 of the bone anchor 14. A delivery instrument may used to engage the closure mechanism 92 with the bone anchor 14 and secure the spinal connection element 12 to the bone anchor 14.

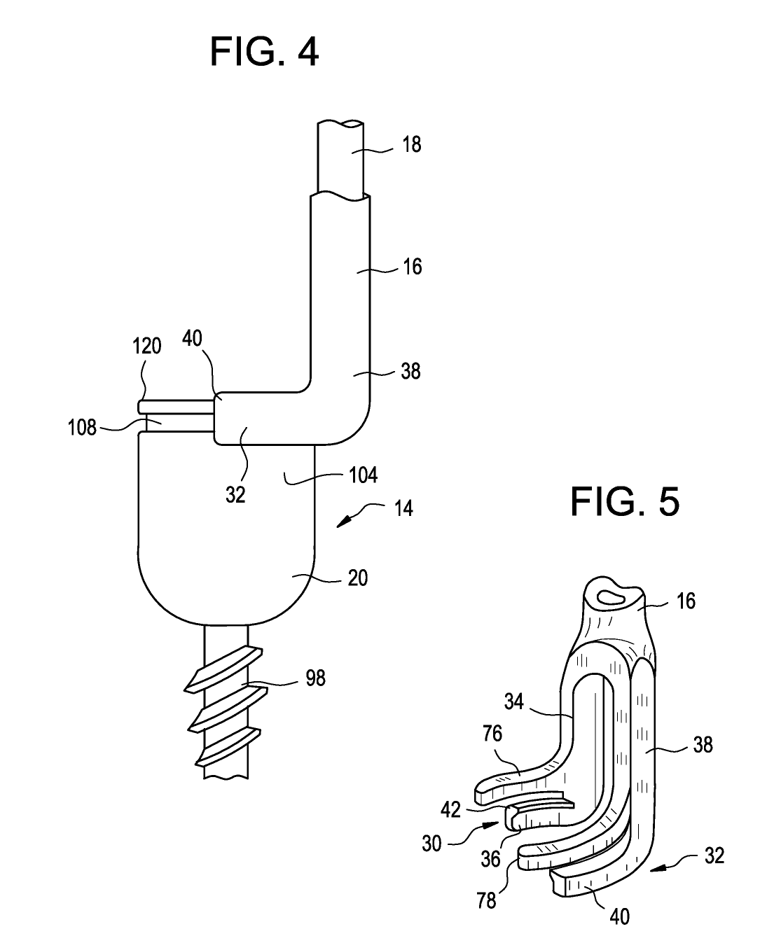

Referring to FIGS. 3 and 4, in certain exemplary embodiments, the distal segments 36, 40 of the first finger 30 and the second finger 32, respectively, may have a length, in a direction parallel to the longitudinal axis L of the first shaft 16, sufficient for the distal segments 36, 40 to extend proximally beyond the top proximal surface 120 of the receiver member 20 of the bone anchor 14. Such a configuration permits the distal segments 36,40 to engage the top proximal surface 120 of the receiver member 20 of the bone anchor 14 when the instrument 10 is engaged to the bone anchor 14. In the exemplary embodiment illustrated in FIGS. 3 and 4, for example, the distal segment 36 of the first finger 30 may include a second projection 62 extending along at least a portion of the distal segment 36 that is spaced apart from the segment 42. The second projection 62 may be positioned to engage the proximal surface 120 of the receiver member 20 of the bone anchor 12 such that a portion of the receiver member 20 is positioned between the projection 42 and the second projection 62. The distal segment 40 of the second finger 32 may include a second projection 64 extending along at least a portion of the distal segment 40 that is spaced apart from the segment 44. The second projection 64 may be positioned to engage the proximal surface 120 of the receiver member 20 of the bone anchor 12 such that a portion of the receiver member 20 is positioned between the projection 44 and the second projection 64.

Referring to FIG. 5, in certain exemplary embodiments the distal end 26 of the first shaft 16 may include a third finger 76 connected and positioned proximal to the first finger 30. The third finger 76 may be engageable with the proximal surface 120 of the receiver member 20 of the bone anchor 14 such that a portion of the receiver member 20 is positioned between the third finger 76 and the first finger 30 when the instrument is coupled to the bone anchor 14. The distal end 26 of the first shaft 16 also may include a fourth finger 78 connected and positioned proximal to the second finger 32. The fourth finger 78 may be engageable with the proximal surface 120 of the receiver member 20 of the bone anchor 14 such that a portion of the receiver member 20 is positioned between the fourth finger 78 and the second finger 32 when the instrument is coupled to the bone anchor 14.

While the instruments and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. An instrument for manipulating a spinal rod relative to a bone anchor, the instrument comprising:
a first shaft having a longitudinal axis,
a first finger and a second finger provided at a distal end of the first shaft, the first finger spaced apart a distance from the second finger, the distance being approximate to an outer diameter of the receiver member of the bone anchor, each of the first finger and the second finger including a projection extending along the length of the finger for engaging a groove provided on the receiver member of the bone anchor; and
a second shaft movable relative to the first shaft in a direction parallel to the longitudinal axis of the first shaft to manipulate the spinal rod relative to the receiver member of the bone anchor.

2. The instrument of claim 1, further comprising an activation mechanism coupled to the first shaft and the second shaft to effect motion of the second shaft relative to the first shaft.

3. The instrument of claim 2, wherein the activation mechanism comprises a first handle connected to the first shaft and a second handle connected to the second shaft.

4. The instrument of claim 3, wherein the first handle and the second handle are oriented generally in a direction perpendicular to the longitudinal axis of the first shaft.

5. The instrument of claim 1, wherein the second shaft is positioned within at least portion of the first shaft and is movable through the portion of the first shaft.

6. The instrument of claim 5, wherein the second shaft threadingly engages the first shaft.

7. The instrument of claim 1, wherein each of the first finger and the second finger includes a second projection extending along the length of the finger, the second projection spaced apart from the first projection.

8. The instrument of claim 1, further comprising a third finger connected and positioned proximal to the first finger and a fourth finger connected and positioned proximal to the second finger.

9. The instrument of claim 1, wherein the first finger and the second finger are flexible and resilient.

10. A spinal implant and instrument kit comprising:
a spinal rod,
a bone anchor having a distal bone engaging portion and a receiving member having a proximal end having a recess for receiving the spinal rod, the receiving member having a substantially U-shaped cross-section defined by two legs separated by the recess, the receiving member having a first groove formed on an exterior surface of the proximal end of a first one of the legs and a second groove formed on an exterior surface of the proximal end of a second one of the legs; and
an instrument for manipulating the spinal rod relative to the bone anchor, the instrument including
a first shaft having a longitudinal axis,
a first finger and a second finger provided at a distal end of the first shaft, the first finger spaced apart a distance from the second finger, the first finger including a first projection extending along at least a portion of the length of the first finger for engaging the first groove provided on the receiver member of the bone anchor, the second finger including a second projection extending along at least a portion of the length of the second finger for engaging the second groove provided on the receiver member of the bone anchor, and
a second shaft movable relative to the first shaft in a direction parallel the longitudinal axis of the first shaft to manipulate the spinal rod relative to the receiver member of the bone anchor.

11. The spinal implant and instrument kit of claim 10, wherein the first finger includes a third projection extending along at least a portion of the first finger, the third projection engageable with a proximal surface of the receiver member such that a portion of the receiver member is positioned between the first projection and the third projection when the instrument is coupled to the bone anchor.

12. The spinal implant and instrument kit of claim 11, wherein the second finger includes a fourth projection extending along at least a portion of the second finger, the fourth projection engageable with a proximal surface of the receiver member such that a portion of the receiver member is positioned between the second projection and the fourth projection when the instrument is coupled to the bone anchor.

13. The spinal implant and instrument kit of claim 10, further comprising a third finger connected and positioned proximal to the first finger, the third finger engageable with a proximal surface of the receiver member such that a portion of the receiver member is positioned between the first finger and the third finger when the instrument is coupled to the bone anchor.

14. The spinal implant and instrument kit of claim 13, further comprising a fourth finger connected and positioned proximal to the second finger, the fourth finger engageable with a proximal surface of the receiver member such that a portion of the receiver member is positioned between the second finger and the fourth finger when the instrument is coupled to the bone anchor.

15. The spinal implant and instrument kit of claim 10, wherein the first finger and the second finger include a proximal segment and a distal segment and the distal segment is oriented at an angle to the longitudinal axis of the first shaft.

16. The spinal implant and instrument kit of claim 15, wherein the distal segment is oriented at an angle greater than approximately 45°.

17. The spinal implant and instrument kit of claim 15, wherein the distal segment is oriented at an angle equal to approximately 90°.

18. A spinal implant and instrument kit comprising:
a bone anchor having a distal bone engaging portion and a receiving member having a proximal end having a recess for receiving the spinal rod, the receiving member having a substantially U-shaped cross-section defined by two legs separated by the recess, the receiving member having a first groove formed on an exterior surface of the proximal end of a first one of the legs and a second groove formed on an exterior surface of the proximal end of a second one of the legs; and
an instrument for engaging the bone anchor, the instrument including
a shaft having a longitudinal axis and a generally U-shaped distal end defined by a first member spaced apart from a second member, at least a portion of the first member and at least a portion of the second member being oriented at an angle to the shaft, the first member including a first projection extending along at least a portion of the length of the first member for engaging the first groove provided on the receiver member of the bone anchor, the second member including a second projection extending along at least a portion of the length of the second member for engaging the second groove provided on the receiver member of the bone anchor.
